(19) United States
(12) Patent Application Publication
(10) Pub. No.: US 2008/0269109 A1
(43) Pub. Date: Oct. 30, 2008

(54) SYSTEM AND METHOD OF RESUSCITATION OF A MAMMAL

(76) Inventor: Lance B. Becker, Philadelphia, PA (US)

Correspondence Address: BELL, BOYD & LLOYD, LLP, P.O. Box 1135, CHICAGO, IL, 60690, US

(21) Appl. No.: 12/112,855

(22) Filed: Apr. 30, 2008

Related U.S. Application Data
(60) Provisional application No. 60/914,992, filed on Apr. 30, 2007.

(51) Int. Cl. A61K 38/28; A61K 31/70; A61P 9/00

(52) U.S. Cl. 514/3; 514/23

(57) ABSTRACT

The present disclosure provides systems and methods of saving people, organs, and cells from the injurious effects of ischemia and the injurious effects of reperfusion by providing artificial circulation while intentionally reducing for a period of rest the normal externally directed functions of the cells.

### PRIORITY CLAIM

The present application claims priority to and the benefit of U.S. Provisional Application No. 60/914,992 filed Apr. 30, 2007, which is being incorporated herein in its entirety by reference.

### BACKGROUND

Most current therapies for recovery of people, organs, and cells following a time interval of ischemia, shock, lack of blood flow, and/or lack of oxygen are based on the well known principal of “immediate reperfusion” which is the current practice of immediately restoring oxygen to an oxygen deprived person, organ or group of cells. This immediate reperfusion or reoxygenation is embodied in the current practice of cardiac resuscitation for victims of sudden temporary death when emergency rescuers or clinicians perform CPR, administer oxygen, provide artificial airways, provide rescue breathing or assisted ventilation, infuse cardio-stimulatory drugs (like epinephrine and others), defibrillate the heart to restore a normal heart beat, and perform other clinical maneuvers to restore the immediate function of the previously ischemic person, organ or group of cells.

### SUMMARY

The present disclosure provides systems and methods of improving resuscitation and minimizing the effects of ischemia and the injurious effects of reperfusion of an individual, organ, or cells that have been exposed to an ischemic condition. In particular, in contrast to known methods that attempt to immediately restore full function of the organ, the systems and methods of the present disclosure include reducing the demand placed on the organ or cells of the organ by other organs and systems and reducing the normal activity of the organs or cells for a sufficient period of time to allow for better restoration of function of the organs or cells after this period.

Accordingly, a method of reversing the effects of an ischemic condition in an organ in a living mammal is provided. The method includes administering an effective amount of a substance to the organ to reduce the external function of the organ and to restore the external function of the organ after a sufficient period of time.

In an embodiment, the substance includes at least one of the agents selected from the group consisting of an agent that alters potassium levels, an agent that alters calcium levels, an agent that reduces activation of cardiac beta receptors, and an agent that reduces mitochondrial electron transport.

In an embodiment, the organ includes a heart, a brain, liver, pancreas, kidney or gastrointestinal organ.

In an embodiment, the method includes administering an effective amount of one or more substances to the organ to optimize the availability of internal energy of the cells of the organ.

In an embodiment, the substance includes at least one of insulin or glucose.

In an embodiment, the method includes providing a supplement for the external function of the organ.

In an embodiment, the supplement includes cardiopulmonary bypass.

In another embodiment, a method of resuscitating a heart of a living mammal is provided. The method includes administering an effective amount of a substance to the heart to reduce effective contraction of the heart. The method also includes providing artificial circulation to the body of the mammal. The method further includes performing clinical maneuvers to restore the effective contraction of the heart after a sufficient period of time.

In an embodiment, the method includes administering an effective amount of one or more substances to the organ to minimize the need for internal energy.

In an embodiment, the substance is a cooling substance.

In a further embodiment, a composition is provided comprising a calcium chelator, a calcium channel blocker, a beta blocker, a mitochondrial inhibitor, an antioxidant, a membrane stabilizing agent and a membrane sealing agent.

In an embodiment, an effective amount of the composition is administered to a subject suffering from cardiac arrest.

An advantage of the present disclosure to improve the likelihood of recovery of a subject, organ, tissue or cells from an ischemic event.

Another advantage of the present disclosure includes reducing the harmful effects of reperfusion of organs, tissues and cells exposed to an ischemic event.

A further advantage of the present disclosure includes providing systems and methods of restoring hemostasis to organs, tissues and cells exposed to an ischemic event prior to requiring a return of the metabolic demands of external function of the damaged organs, tissues and cells.

Additional features and advantages are described herein, and will be apparent from, the following Detailed Description.

### DETAILED DESCRIPTION

The present disclosure provides systems and methods of reversing the effects of damage to organs, tissues and cells of a mammal. The methods include reducing for a period of time the function of at least one organ exposed to ischemia before stimulating the organ to resume its normal function. In particular, an effective amount of one or more substances is administered to a mammal to reduce the performance of external functions of cells and organs of the mammal. The external functions of cells and organs of the mammal are reduced for a period of time sufficient to enable the cells to more rapidly recover from any abnormal conditions or states that have developed or occurred during the ischemia period and to restore the homeostatic mechanisms of the cell to substantially normal so that, after this period, the person, organ, and cells can resume external function without suffering further injurious effects of uncontrolled reperfusion.

Cellular energy (or work) can be directed toward internal energy of the individual cells of an organ and external function of the organ. Energy is required to maintain the basic viability of the cell required for the life of the single cell or internally required energy. Another large part of the cellular energy pool is directed toward the external function or “community work” of the organ that benefits the organism as a whole. This energy, in turn, may provide minimal direct benefit to the specific cells performing this external function.

The present disclosure includes decreasing the level of community work required from vulnerable injured cells for a temporary period to allow intracellular processes to repair rather than promote cell death pathways. Therefore, instead of using methods to immediately restart the normal activity of the organ that has suffered from ischemia, such as the normal contraction of the heart, the treatment of the present disclosure is directed to creating conditions in which the injured cells can stabilize and at least begin to recover prior to stimulating the organ to resume its normal activity.

The treatment of the present disclosure may be accomplished in an organ-specific manner with protection for organs and tissues such as the brain, heart, and endothelium as initial targets of the present disclosure. For example, the present disclosure and agents may be used to reverse the effects of ischemia on the heart, brain, liver, pancreas, kidney, GI system, and other organs for specific organ resuscitation after a period of ischemia. The cells of these organs are specialized for performing the external function of the organ such as muscle cells which contract and perform mechanical work, brain cells which depolarize and conduct electrical currents along a specific path, liver cells which metabolize and synthesize molecules and perform other external functions of the liver, kidney cells which perform the work of the kidney, and so on.

Therefore, in an embodiment, of the present disclosure, a subject suffering from cardiac arrest is rapidly placed on cardiopulmonary bypass (CPB) for circulatory support while enabling organs such as the heart and brain to undergo a rest and repair time period and to be relieved from performing community work before restarting the organs to resume community work without suffering additional reperfusion injury.

In an embodiment, the present disclosure includes causing the organs and cells to diminish or halt their external function or to reduce or halt their normal function. In particular, the present disclosure may include preventing the contraction of the heart with agents specifically administered to prevent or reduce contraction and external mechanical work of pumping blood by the heart. To reduce these functions, various agents or combinations of agents are administered including agents involved in preventing normal depolarization of the conduction and mechanical systems of the cardiomyocytes. In addition to agents sufficient and effective to reduce/stop normal heart and brain activity, agents may be administered that prevent further cellular injury, provide energy substrates, provide cellular protection, and prevent mitochondrial dysfunction with initiation of death signaling.

In an embodiment, a subject or individual identified as having an ischemic condition such as cardiac arrest is administered an effective amount of one or more agents sufficient to halt contraction of the heart. Ischemia or ischemic condition may include any lack of an adequate supply of blood, oxygen or other vital nutrients to cells of a mammal. Ischemia may result from conditions such as inadequate perfusion, shock, lack of oxygen and the like and may cause sudden temporary death.

As referred to herein, an effective amount of a substance may include an amount sufficient to reduce the external function of an organ. A substance may include a chemical, drug, biological or any other suitable agent. The substance may be administered to the body, organ, tissue, cell or any other suitable target in any suitable manner such as systemically. The substance may be administered directly to the target and, in an embodiment, the administration of the substance may be substantially isolated to that target.

In an embodiment of the present disclosure, distinct substances or agents or combinations of agents are administered to a subject suffering from ischemia or an ischemic event. Such agents may include agents involved in altering potassium levels and/or calcium levels such as potassium and calcium channel blockers, agents involved in reducing activation of cardiac beta receptors such as beta blockers, and agents involved in reducing mitochondrial electron transport. In addition, myosin inhibitors such as 2,3-butanedione monoxime that block ATP and calcium binding to actin-myosin may be administered to the subject. Substances such as adenosine and lidocaine may also be used to block ATP binding to actin-myosin. Also, solutions such as a cardioplegic solution or other solutions capable of hyperpolarizing or stabilizing cellular membranes, scavenging free-radicals and other cell protective functions may be administered.

In an embodiment three types of therapies are provided to the subject including a “damage control and rescue” therapy, a “maintenance and healing” therapy and a “return to function” therapy. In an embodiment, the “damage control and rescue” therapy is the agent or combination of agents initially administered to a subject who has suffered an ischemic event. In an embodiment, the “damage control and rescue” therapy is the agent or combination of agents initially administered to a subject who has suffered an ischemic event. In an embodiment, the “maintenance and healing” therapy is administered after the “damage control and rescue” therapy. In an embodiment, the “return to function” therapy is administered to the subject after the “maintenance and healing” therapy upon a determination that the condition of the cells exposed to the ischemic event have improved. In an embodiment, therapies are administered to a subject according to a predetermined sequence. It should be appreciated, however, that the therapies may be administered alone, together, in any sequence or as needed. The therapies may be delivered by any suitable route of administration including intravenous or intra-arterial lines introduced into the subject. In an embodiment, the “damage control and rescue” therapy is administered to a subject as an initial rescue stasis hibernation therapy in conjunction with the initiation of CPB.

In an embodiment, the therapies of the present disclosure include administering an effective amount of one or more substances capable of protecting cells from calcium disturbance, oxidant burst/surges apoptosis, programmed cell death, and proteolysis, protecting the cell membrane, preventing “community work” by vulnerable injured cells and providing substrate for energy and biosynthesis work.

The present disclosure includes administering an effective amount of one or more substances capable of providing protection from calcium disturbance. A rapid rise in calcium has been found to be associated with necrosis, apoptosis, and all cell death pathways. In an embodiment, the initial therapy substantially excludes calcium in the solution. In addition, the initial therapy may include calcium chelators, such as EDTA, and BATPHA, calcium channel blockers, and other suitable agents effective to minimize the actions of calcium. Calcium channel blockers may include for example, amlopidine, diltiazem, isradipine, nifedipine, nicardipine, and verapamil at any suitable dose. For example, nifedipine may be administered at a dose of about 4 mcg/kg and within a range of about 10 mcg/kg to about 4 mg/kg. It should be appreciated that the components may be administered individually or combined into a single therapy.

The present disclosure includes administering an effective amount of one or more substances capable of providing protection from oxidant burst/surges to reduce the flow of electrons through the sites of electron transport that transfer electrons to oxygen to temporarily reduce mitochondrial respiration. Energy production within the mitochondria is a delicately balanced series of electron transfers from one protein to the next in an orderly fashion. Disruption of this carefully balanced electron flow produces excessive free radicals. Excessive free radicals at low levels first signal the cell to repair the imbalance. However, if this repair is not successful or if the free radical production reaches higher levels the signal for repair changes into a feed forward positive feedback amplification that calls for cell death in the presence of oxygen. Reperfusion injury results when the mitochondria sense an overwhelming message of death via the biochemical alterations of excessive electron flow and free radical production in the presence of high molecular oxygen levels in the cell. A rapid rise in reactive oxygen species (ROS) is associated with reperfusion in the first minutes of ischemia. Therefore, the present disclosure includes administering agents that reduce cell death signals and, in particular decrease electron flow through mitochondria and free radical production, decrease molecular oxygen while avoiding community work which is also powered by the mitochondrial ATP. To this end, the present disclosure includes providing agents to reduce ROS formation and to neutralize/detoxify excessive ROS generation. Alternatively, or in addition, in an embodiment, ROS generation is reduced in reperfusion with hypothermia coupled, in an embodiment, with gas phase mitochondrial inhibitors for rapid action (CO₂, NO, CO, HS, CN). In an embodiment, the present disclosure includes administering an effective amount of one or more substances capable of decrease ROS generation from one or more of Sites I, III and IV of mitochondria using, for example, pharmacological inhibitors specific for such sites such as glyceollin, rotenone, N-methyl-4-phenylpyridinium and sodium hydrogensulfide may be used at any suitable dose. For example, the dose of sodium hydrogensulfide may be from about 0.1 micro mol/kg to about 1000 micro mol/kg.

The present disclosure includes administering an effective amount of one or more substances capable of providing protection from apoptosis, programmed cell death, and proteolysis. Several apoptotic cell death pathways are reported activated following cardiac arrest. Most of these apoptotic pathways seem to involve the initiation of caspases, calpains (due to elevated calcium), and proteolytic enzymes that promote the breaking down of the cell. The present disclosure includes the use of agents to neutralize this death process such as calpain inhibitors such as Acetyl-Leu-Leu-Norieucinal (20 μM), N-Acetyl-Leu-Leu-Methioninal (100 μM, cell-permeable chelators of intracellular calcium stores such as BAPTA-AM (10 μM), agents chelating extracellular calcium such as EGTA (10 mM).

The present disclosure includes administering an effective amount of one or more substances capable of providing protection to the cell membrane. Events of ischemia reperfusion result in cell membrane blebbing and disruption. Cell membranes may be able to be repaired if a temporary cell membrane “sealant” is provided such as polaximer-188, calcium 2-ethylamino phosphate, polyethylene glycol or any other suitable membrane protectant.

The present disclosure includes administering an effective amount of one or more substances capable of providing substrate for energy and biosynthesis work such as glycolyticly-derived energy (ATP), insulin, potassium and glycolytic substrates. For example, substrates that favor rapid glycolytic ATP production over respiration for ATP in the immediate post ischemic period such as Fructose 1-6 bisphosphate, ATP-Mg—Cl, glutamine transporters, Glucose-Insulin-K and the like may be used.

The present disclosure also includes administering an effective amount of one or more substances capable of providing general cytoprotection to protect cells from ischemia and reperfusion. These substances may include agents such as estrogen, adenosine, opioids, bradykinins, erythropoietin, beta-estradiol and 17-alpha-estradiol at any suitable dose. For example, the dose of 17-alpha-estradiol dose may be from about 10 micro mol/kg to about 2000 mmol/kg single dose.

Prior to, during or after the present disclosure are initiated, resuscitation of a mammal is directed toward immediate support of circulation with artificial (external and mechanical) means in addition to, in an embodiment administration of agents to prevent full external work functions in the cells. This includes allowing the heart to stop beating on its own, decreasing or preventing resumption of heart beat. Therefore, no further attempts are made to defibrillate the heart or to administer cardio-stimulatory agents or neuro-stimulatory agents.

In an embodiment, alternative or artificial methods may be used to supplement or replace the external function of the organ. For example, if the function of the heart is temporarily reduced or stopped, circulation of the blood flow to other organs may be maintained using an artificial source of circulation in combination with the cessation of organ and cellular external work. It should be appreciated that immediate support of circulation may include external CPR used as a bridge until adequate blood flow is assured using an external artificial device. Artificial blood flow or artificial circulation of a fluid through at least a portion of the circulatory system may be provided using external devices for circulation, such as a LUCAS device or the like. Internal circulatory devices such as temporary cardiopulmonary bypass machine, a heart-lung machine or any other suitable artificial source of circulation may also be used to provide blood flow to the arrested patient. A particular advantage of CPB is the ability to control gas proportions and add drugs rapidly. This can be rapidly performed in the hospital or emergency department, and may be possible to initiate even earlier in the treatment of the subject. For example, cannulation of the femoral vein and artery with placement of large bore catheters connected to a circulatory pump may achieve the required blood flow for most subjects. Other suitable devices may be used for artificial support of circulation.

To reduce the effects of ischemia on the brain after ischemia, the present disclosure include maintaining proper circulatory support through either normal heart beat and blood pressure if the heart is beating, or through external circulatory support such as CPB if the heart function is reduced. In addition, the methods of the present disclosure include administering agents to temporarily reduce or provide a respite for the normal depolarization and electrical currents of the brain and nervous system. In the brain, for example, substances such as NMDA blockers, such as amantadine, dextromethorphan, dextrorphan, dizocilpine, ibogaine, ketamine, nitrous oxide phencyclidine, riluzole, tiletamine, aptiganel, memantine remacimide, 7-chlorokynurenate′ 5,7-dichlorokynurenic, 2-amino-7-phosphonoheptanoic acid, R-2-amino-5-phosphonopentanoate, 3-[(R)-2-carboxypiperazin-4-yl]-prop-2-enyl-1-phosphonic acid and sodium channel blockers, such as tetrodotoxin and lidocaine, may be used to protect neural cells from depolarization. The present disclosure also includes administering agents or combinations of agents capable of preventing brain injury and preserving potentially viable neurons and subsequent neuronal function in patients who suffer ischemia to the whole or part of the brain. For example, the present disclosure includes administering agents or combinations of agents capable of preventing calcium overload such as low calcium solutions, calcium chelators, and calcium channel blockers. Additional agents may be administered rapidly including rapid induction of hypothermia, mitochondrial inhibitors, antioxidants, membrane/lipid stabilizing agents, membrane sealing agents such as polaximer-188, and other agents to prevent the metabolic pathways of cell death, excitotoxicity, and to allow neuronal tissue an opportunity to rest from normal neuronal function. During this time the homeostatic and restorative pathways continue to function to correct cellular abnormalities that were created during the period of ischemia.

As referred to herein, a sufficient period of time includes the time required to improve the response of the cells of the organ to the ischemic condition. A sufficient period of time may include the time required to begin to reverse the abnormal conditions of the cell incurred during the ischemic event and to improve the homeostatic mechanisms of the cell. For example, the present disclosure may include allowing the time necessary to reduce oxygen uptake, slow metabolism and adjust blood chemistry for gradual and safe reperfusion.

To prevent the temporary resumption of spontaneous heart beat prior to completion of an optimal rest period of the damaged cells, one or more agents may be administered to the subject, either alone or in combination, such as potassium, calcium binders and calcium chelators, beta blockers, agents to prevent muscle contraction such as 2,3-butadione monoxime, and specific mitochondrial inhibitors.

In various embodiments, agents may be administered at a temperature that reduces the requirements of the cells of an organ such as a temperature of less than about 37° C. Other suitable methods of cooling the organ may also be used. In some embodiments, the target organ may be cooled to a temperature of about 33° C. In an embodiment, a cooled saline solution is administered to reduce the external function of an organ, such as a cool saline ice slurry disclosed in U.S. Pat. No. 6,547,811. In an embodiment, hypothermia is induced in patient as rapidly as is feasible, optimally during the intra-arrest period while the heart is stopped.

In an embodiment, a “maintenance and healing” therapy or maintenance therapy is administered to a subject. In an embodiment the maintenance therapy includes an effective amount of one or more substances administered to the mammal to optimize the availability of internal energy required for the cell to survive. Such substances may include agents that improve maintenance of ionic gradients of sodium, potassium, calcium and other ions, maintenance of lipid membrane integrity, maintenance of mitochondrial health, and prevention of acute generalized cell death via apoptosis, necrosis, activation of caspases, activation of calpains, and other mechanisms of cell death. In addition, the present disclosure includes administering an effective amount of one or more substances capable of augmenting energy produced via glycolysis to provide additional energy which is targeted toward internal (non-external work). For example, in an embodiment, an effective amount of insulin and glucose or any other suitable agent that provides additional energy targeted toward internal energy is administered to an individual suffering from an ischemic condition.

Following a sufficient period of time to allow the subject, organs, and cells to begin to slow or reverse the harmful effects of the ischemic insult, the present disclosure includes restoring the external function of the organ. The present disclosure includes assessing indications of normalizing function of the organ, tissue or cells of the subject to determine when to restore the external function of the organ. For example, normal function of the rested body, organ, tissue or cell may be restored when intracellular calcium overload does not occur with resumption of external work or function. Normal function of the rested body, organ, tissue or cell may be restored when cytochrome C and other mitochondrial membrane proteins are not being actively released and cytochrome C and other mitochondrial membrane proteins are not further released upon resumption of function and external work. Normal function of the rested body, organ, tissue or cell may be restored when oxidant generation and reactive oxygen species are not being generated in higher deleterious quantities and oxidants and reactive oxygen species are not produced in deleterious quantities with resumption of external work and restoration of electron transport function. In addition, resumption of external work may occur safely when mitochondrial ATP levels return to normal and do not fall to low levels with performance of external work.

Additional indicators for restoring external function and initiating the present disclosure for resuscitation may include when a heart beat is unable to be restored, when traditional ACLS fails to return a stable pulse, or when the time in cardiac arrest is sufficiently long such that results from this method are superior to standard ACLS. It should be appreciated that the time required for cellular recovery may depend on the extent of the ischemia prior to initiation of the treatment of the present disclosure.

The resumption of normal activity may occur spontaneously when the effects of administered agents begin to wear off or when the effects are specifically reversed using additional agents specifically designed to reverse the resting (non-externally working) state. In an embodiment, a “return to function” therapy or restoration therapy is administered to a subject. In an embodiment, the restoration therapy includes performing advanced cardiac life support protocols such as restoring oxygen to an oxygen deprived person, organ or group of cells, administering cardiopulmonary resuscitation, administration of oxygen, establishing artificial airways, providing rescue breathing or assisted ventilation, infusing cardio-stimulatory drugs such as epinephrine, defibrillation of the heart to restore effective contraction of the heart in the form of a normal heart beat, and other clinical maneuvers to restore the immediate function of the previously ischemic person, organ or group of cells. In the out-of-hospital setting and in the in-hospital setting, traditional rescue therapies with ACLS as currently practiced may be initiated to restart the heart. Certain agents may be required to correct calcium, and other ions. Certain agents may be required to antagonize beta blockers or other channel blockers. Certain agents may be required to antagonize mitochondrial inhibitors.

In an embodiment, the treatment of the present disclosure is initiated instead of standard CPR in a subject suffering from cardiac arrest for more than five minutes. In an embodiment, the treatment of the present disclosure is initiated in a subject suffering from cardiac arrest up to and exceeding fifteen minutes after the onset of cardiac arrest. At certain time periods after the onset of cardiac arrest, survival rates and improved organ function are consistently better using the treatment of the present disclosure rather than standard current ACLS-style resuscitation consisting of CPR, defibrillation, and advanced drug therapies. It should be appreciated that the treatment of the present disclosure may be administered after failed attempts with these standard resuscitative techniques. In an embodiment, at least one of the therapies is administered early in cardiac arrest in conjunction with implementation of CPB. At least one of the therapies of the present disclosure may be administered while the heart is not beating, with CPR being performed, or as CPB is first initiated. In an embodiment, the therapy is administered as one or more components of the priming solution of the CPB device.

The following example is illustrative of the present disclosure and includes a series of actions which may or may not be performed one or more times and which may be performed in any suitable sequence. Accordingly, the following example is not intended to limit the present disclosure in any way.

### EXAMPLE

1. A patient is identified to be in cardiac arrest or suffering from another severe shock state (which could include hemorrhage or sepsis) wherein active metabolic pathways in the cells and organs are leading to additional cell death, cell injury, tissue injury, and organ dysfunction.

2. An initial “damage control and rescue” therapy is administered to the patient systemically or perfused into vital organs to decrease external work performed by organs at risk for ischemic damage such as the brain and heart. The initial therapy includes a mixture of a beta-blocker (e.g., esrmolol), 2,3-butanedione monoxime at a dose of 1 micro mol/kg to 50 mmol/kg to prevent cross-bridging and muscular contraction, and adenosine, lidocaine, or other channel blocking agents such as calcium blocking agents.

3. Artificial circulation is started just before or just after the mixture above is administered to the patient to relieve vital organs of their normal level of external work and to prevent irreversible death of the patient. This artificial form of circulation is provided by an external device designed to provide blood flow support (like the LUCAS device or the Autopulse devise), or alternatively CPB is instituted rapidly.

4. Agents to control mitochondrial respiration and free radical generation are administered to the patient. These agents include gases like CO2, NO, CO, H2, and other small molecules that will bind to the respiratory enzymes to diminish electron flow through the electron transport chain. Other mitochondrial inhibitors could include uncoupling agents, as well as specific inhibitors of the major electron transport complexes.

5. As soon as feasible, systemic cooling is performed to additionally provide neurological and tissue protection against ischemia and reperfusion. This cooling is typically administered to lower body temperature by at least 2° C. and will often target a temperature of less than 34° C.

6. Agents to protect the cell membrane wall from loss of integrity (e.g., polaximar-188) are administered to both protect cell membranes from developing leakages and to repair leaking membranes that developed during ischemia.

7. Specific neuroprotective agents are administered to prevent neuronal injury. Drugs to prevent excitotoxicity include chelators, calcium channel blockers, sodium channel blockers, sodium/calcium exchange inhibitors, and potassium channel blockers.

8. This “resting” condition is maintained until there is evidence that normal cellular function can be safely restarted without additional injury to cells or tissues. This “resting” condition is maintained up to sat least ten minutes and may require more than 24 hours for full cellular recovery to occur.

9. After ischemic injury has been substantially stabilized or reversed based on physiological and/or biochemical indicators of improved condition of the cells, tissues and organs of the patient, the administration of agents to depress external work (i.e., mitochondrial inhibitors, channel blockers, and blockers of external function) are gradually discontinued or allowed to metabolize.

10. Active cooling is stopped and the patient is gradually warmed to near normal temperatures.

11. As the resuscitative treatment of the present disclosure is withdrawn, standard intensive care unit and critical care therapies are initiated to return organs to full functioning.

It should be understood that various changes and modifications to the present disclosure described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of reversing the effects of an ischemic condition in an organ in a living mammal comprising:
administering an effective amount of a substance to the organ to reduce the external function of the organ; and
restoring the external function of the organ after a sufficient period of time.

2. The method of claim 1, wherein the substance includes at least one of the agents selected from the group consisting of an agent that alters potassium levels, an agent that alters calcium levels, an agent that reduces activation of cardiac beta receptors, and an agent that reduces mitochondrial electron transport.

3. The method of claim 1, wherein the organ includes a heart, a brain, liver, pancreas, kidney or gastrointestinal organ.

4. The method of claim 1, which includes administering an effective amount of one or more substances to the organ to optimize the availability of internal energy of the cells of the organ.

5. The method of claim 4, wherein the substance includes at least one of insulin or glucose.

6. The method of claim 1, which includes providing a supplement for the external function of the organ.

7. The method of claim 6, wherein the supplement includes cardiopulmonary bypass.

8. A method of resuscitating a heart of a living mammal comprising:
administering an effective amount of a substance to the heart to reduce effective contraction of the heart;
providing artificial circulation to the body of the mammal; and
perform clinical maneuvers to restore the effective contraction of the heart after a sufficient period of time.

9. The method of claim 8, wherein the substance includes at least one of the agents selected from the group consisting of an agent that alters potassium levels, an agent that alters calcium levels, an agent that reduces activation of cardiac beta receptors, and an agent that reduces mitochondrial electron transport.

10. The method of claim 8, which includes administering an effective amount of one or more substances to the organ to optimize the availability of internal energy of the cells of the organ.

11. The method of claim 10, wherein the substance includes at least one of insulin or glucose.

12. The method of claim 8, wherein the supplement includes cardiopulmonary bypass.

13. The method of claim 8, which includes administering an effective amount of one or more substances to the organ to minimize the need for internal energy.

14. The method of claim 13, wherein the substance is a cooling substance.

15. A composition comprising:
a calcium chelator;
a calcium channel blocker;
a beta blocker
a mitochondrial inhibitor;
an antioxidant;
a membrane stabilizing agent; and
a membrane sealing agent.

16. The composition of claim 15, wherein an effective amount of the composition is administered to a subject suffering from cardiac arrest.

17. A method of treating an ischemic condition comprising:
administering an effective amount of a first substance to a subject suffering from the ischemic condition, wherein the first substance reduces the external function of an organ affected by the ischemic condition;
administering an effective amount of a second substance to the subject, wherein the second substance optimizes the availability of internal energy of the cells of the organ; and
restoring the external function of the organ after a sufficient period of time.

18. The method of claim 17, wherein the first substance includes at least one of the agents selected from the group consisting of an agent that alters potassium levels, an agent that alters calcium levels, an agent that reduces activation of cardiac beta receptors, and an agent that reduces mitochondrial electron transport.

19. The method of claim 17, wherein the second substance includes at least one of insulin or glucose.

20. The method of claim 17, which includes providing circulatory support to the subject.
